(19) United States
(12) Patent Application Publication
(10) Pub. No.: US 2003/0231980 A1
(43) Pub. Date: Dec. 18, 2003

(54) Germ cleanable pull cord

(76) Inventor: John S. Jarmain, Germantown, WI (US)

Correspondence Address: Donald J. Ersler, 725 Garvens Avenue, Brookfield, WI, 53005, US

(21) Appl. No.: 10/172,861

(22) Filed: Jun. 18, 2002

(51) Int. Cl. A61L 2/00

(52) U.S. Cl. 422/28; 422/1; 422/292

(57) ABSTRACT

A germ cleanable pull cord includes an inner layer coated with an outer layer. Preferably, one end of the germ cleanable pull cord is knotted and retained in a call box or the like. The outer layer must have a smooth exterior surface free from ridges, pits, projections, or other surface irregularities to prevent germs from being retained thereupon after cleaning. The outer layer must also be able to endure multiple cleanings with a germicidal or antiseptic cleaning medium.

### BACKGROUND OF THE INVENTION

1. Field of the Invention

The present invention relates generally to pull cords for call boxes used in hospitals and more specifically to a germ cleanable pull cord which may be thoroughly cleaned with a germicide or antiseptic cleaning medium.

2. Discussion of the Prior Art

Pull cords for call boxes or the like used in hospitals are typically fabricated from a fine metal chain or a nylon cord. The call boxes are used by patients in a hospital to summon medical assistance. The fine metal chain cannot be fully cleaned from germs due to unreachable areas inside the links. The nylon cord cannot be fully cleaned from germs due to the inner fibers. Germs can be spread to a pull cord by hand contact, air borne contact, condensation, or through other types contamination. An additional source that retains germs in a hospital is not welcome. Pull cords fabricated from a fine metal chain or nylon are a potential source of germs when they cannot be fully cleaned.

Accordingly, there is a clearly felt need in the art for a germ cleanable pull cord which is capable of being fully cleaned from germs.

### SUMMARY OF THE INVENTION

The present invention provides a germ cleanable pull cord that is capable of being fully cleaned from germs. The germ cleanable pull cord includes an inner cord coated with an outer layer. The outer layer must have a smooth exterior surface free from ridges, pits, projections, or other surface irregularities to prevent germs from being retained thereupon after cleaning. Preferably, one end of the germ cleanable pull cord is knotted and retained in a call box or the like. Preferably, a pull knob is retained on the other end of the germ cleanable pull cord by inserting thereof through a hole in pull knob and knotting the other end of the germ cleanable pull cord. The smooth outer layer must be capable of enduring cleaning with a germicidal or antiseptic cleaning medium.

Accordingly, it is an object of the present invention to provide a germ cleanable pull cord which may be thoroughly cleaned from germs with a germicidal or antiseptic cleaning medium.

These and additional objects, advantages, features and benefits of the present invention will become apparent from the following specification.

### BRIEF DESCRIPTION OF THE DRAWINGS

FIG. 1 is a perspective view of a germ cleanable pull cord extending from a call box in accordance with the present invention.

FIG. 2 is an enlarged cross sectional view of a germ cleanable pull cord in accordance with the present invention.

FIG. 3 is an enlarged view of one end of a germ cleanable pull cord attached to an activation switch of a call box in accordance with the present invention.

FIG. 4 is an enlarged view of a knotted germ cleanable pull cord retained by a pull knob in accordance with the present invention.

### DETAILED DESCRIPTION OF THE PREFERRED EMBODIMENTS

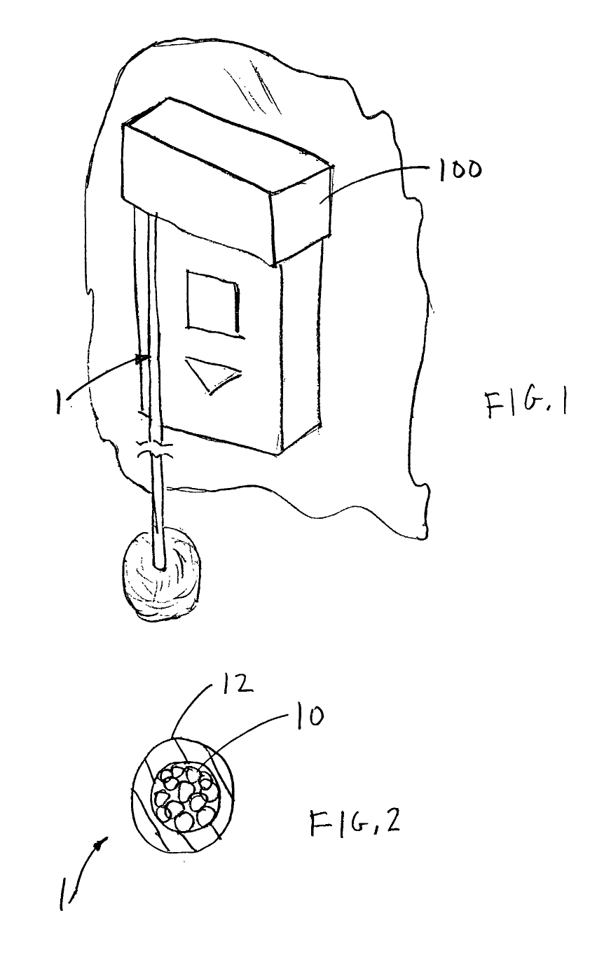

With reference now to the drawings, and particularly to FIG. 1, there is shown a perspective view of a germ cleanable pull cord 1 attached to a call box 100. With reference to FIG. 2, the germ cleanable pull cord 1 includes an inner cord 10 coated with an outer layer 12. Preferably, the inner cord 10 is fabricated from a nylon material, similar to that presently used for pull cords on hospital call boxes. Preferably, the outer layer 12 is fabricated from a moldable plastic material. However, other materials could also be used. Alternatively, the outer layer 12 may be fabricated from a material which contains a germicide. The germicide may partially or completely eliminate the need for cleaning with a germicidal or antiseptic cleaning medium. The outer layer 12 must have a smooth exterior surface free from ridges, pits, projections, or other surface irregularities to prevent germs from being retained thereupon after cleaning. The outer layer 12 must be able to endure multiple cleanings with a germicidal or antiseptic cleaning medium. The germicidal or antiseptic cleaning medium is preferably a germicidal or antiseptic cleaning solution.

The color of the outer layer 12 is preferably white, but other colors may also be used. As an added safety feature, the outer layer 12 would be fabricated from a clear material and the inner cord coated or impregnated with a litmus substance. If the clear outer layer 12 would crack, the inner cord 10 would change color from white to another color when a germ contacts the litmus substance.

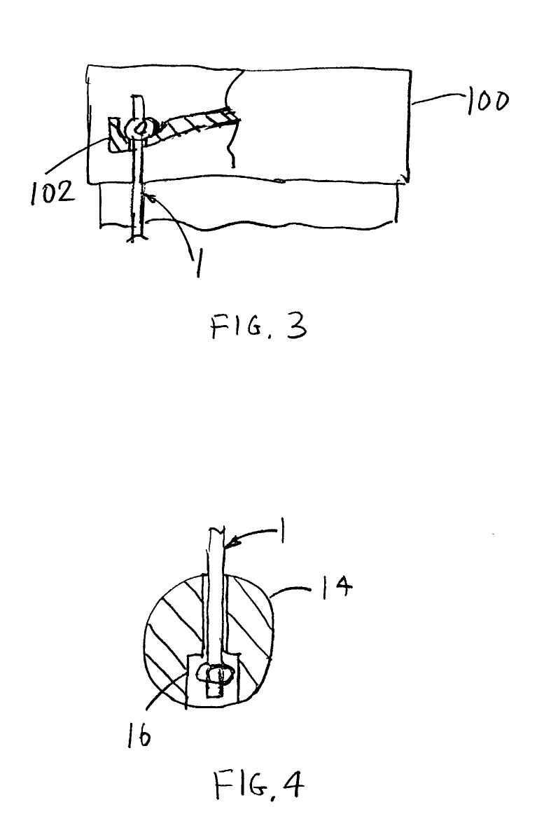

With reference to FIG. 3, one end of the germ cleanable pull cord 1 is knotted and retained in an activation switch 102 of a call box 100 or the like. However, other methods of retaining the germ cleanable pull cord 1 may also be used. With reference to FIG. 4, a pull knob 14 is attached to the other end of the germ cleanable pull cord 1. Preferably, a counterbored hole 16 is formed in the pull knob 14. The other end of the germ cleanable pull cord 1 is inserted through a back entrance of the counterbored hole 16 and knotted. However, other methods of retaining the pull knob 14 may also be used, such as molding the pull knob 14 on to the other end of the germ cleanable pull cord 1. The pull knob 14 must be fabricated from a material which is able to endure multiple cleanings with a germicidal or antiseptic cleaning medium.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

I claim:

1. A germ cleanable pull cord comprising:
an inner cord; and
an outer layer being applied to said inner cord, said outer layer having a smooth exterior surface to prevent germs from being retained thereupon after cleaning with a germicide or antiseptic cleaning medium, said outer layer being able to endure multiple cleanings with the germicide or antiseptic cleaning medium.

2. The germ cleanable pull cord of claim 1 wherein:
said inner cord being fabricated from a nylon material.

3. The germ cleanable pull cord of claim 1 wherein:
said outer layer being fabricated from a moldable plastic.

4. The germ cleanable pull cord of claim 1 wherein:
said outer layer being fabricated from a material which contains a germicide.

5. A method of decreasing the probability of obtaining germs from a call box, comprising the steps of:
providing the call box with an activation switch;
providing an inner cord;
applying an outer layer to said inner cord to form a pull cord, said outer layer having a smooth exterior to prevent the retention of germs after cleaning with a germicidal or antiseptic cleaning medium; and
attaching one end of said pull cord to said activation switch.

6. The method of decreasing the probability of obtaining germs from a call box of claim 5, further comprising the step of:
attaching the other end of said pull cord to a pull knob.

7. The method of decreasing the probability of obtaining germs from a call box of claim 6, further comprising the step of:
fabricating said pull knob from a material which endures multiple cleanings with the germicide or antiseptic cleaning medium.

8. The method of decreasing the probability of obtaining germs from a call box of claim 5, further comprising the step of:
fabricating said outer layer from a material which endures multiple cleanings with the germicide or antiseptic cleaning medium.

9. The method of decreasing the probability of obtaining germs from a call box of claim 5, further comprising the step of:
fabricating said outer layer from a moldable plastic.

10. The method of decreasing the probability of obtaining germs from a call box of claim 5, further comprising the step of:
fabricating said inner cord from a nylon material.

11. The method of decreasing the probability of obtaining germs from a call box of claim 5, further comprising the step of:
fabricating said outer layer from a material which contains a germicide.

12. A germ cleanable pull cord in combination with a call box, comprising:
an inner cord; and
an outer layer being applied to said inner cord to form said cleanable pull cord, said outer layer having a smooth exterior surface to prevent germs from being retained thereupon after cleaning with a germicide or antiseptic cleaning medium, attaching one end of said germ cleanable pull cord to an activation switch of the call box.

13. The germ cleanable pull cord in combination with a call box of claim 12, further comprising:
a pull knob being attached to the other end of said germ cleanable pull cord.

14. The germ cleanable pull cord in combination with a call box of claim 13, further comprising:
said pull knob being fabricated from a material which endures multiple cleanings from a germicide or antiseptic cleaning medium.

15. The germ cleanable pull cord in combination with a call box of claim 12, further comprising:
said outer layer being fabricated from a material which endures multiple cleanings from a germicide or antiseptic cleaning medium.

16. The germ cleanable pull cord in combination with a call box of claim 12, further comprising:
said outer layer being fabricated from a moldable plastic.

17. The germ cleanable pull cord in combination with a call box of claim 12, further comprising:
said inner cord being fabricated from a nylon material.

18. The germ cleanable pull cord in combination with a call box of claim 12, further comprising:
said outer layer being fabricated from a material which contains a germicide.
